(19) United States
(12) Patent Application Publication
(10) Pub. No.: US 2023/0248869 A1
(43) Pub. Date: Aug. 10, 2023

(54) METHODS, SYSTEMS, AND DEVICES FOR PROVIDING LIGHT FOR SURFACE DISINFECTION

(71) Applicant: Actev Motors, Inc., Mooresville, NC (US)

(72) Inventors: David Bundy Bell, Mooresville, NC (US); Matthew Elwin Bell, Cornelius, NC (US); Timothy Robert Bell, Huntersville, NC (US)

(21) Appl. No.: 18/165,831

(22) Filed: Feb. 7, 2023

Related U.S. Application Data
(63) Continuation of application No. PCT/US23/62133, filed on Feb. 7, 2023.
(60) Provisional application No. 63/372,031, filed on Feb. 9, 2022.

(51) Int. Cl. A61L 2/24; A61L 2/10

(52) U.S. Cl. CPC A61L 2/24; A61L 2/10; A61L 2202/14; A61L 2202/11

(57) ABSTRACT

Disclosed herein are methods, systems, and devices for providing light for surface disinfection. In one embodiment, a device includes a controller, a disinfecting light source electronically coupled with the controller, and a positioning mechanism mechanically coupled with the light source and electronically coupled with the controller. Additionally, the controller is configured to direct the disinfecting light source using the positioning mechanism.

### PRIORITY CLAIM

This application is a continuation application of PCT Patent Application Serial No. PCT/US2023/062133 filed Feb. 7, 2023, entitled “METHODS, SYSTEMS, AND DEVICES FOR PROVIDING LIGHT FOR SURFACE DISINFECTION,” which claims priority to U.S. Provisional Patent Application Ser. No. 63/372,031 filed Feb. 9, 2022, entitled “FOCUSED ULTRAVIOLET LIGHT PROJECTOR FOR SURFACE DISINFECTION.” The disclosures of the aforementioned applications are incorporated herein by reference in their entireties.

### TECHNICAL FIELD

The present invention relates generally to surface disinfection using light sources and more specifically to stationary devices having computer controlled light sources that may be directed manually or automatically directed toward identified objects for surface disinfection.

### BACKGROUND

Ultraviolet-C (UVC) ultraviolet disinfection systems have become popular in hospitals for the reduction of health care-associated infections (HAIs). Millions of patients and staff are affected by HAIs each year. These UVC ultraviolet disinfection systems broadcast high-intensity UVC light to permanently damage the ribonucleic acid (RNA) and deoxyribonucleic acid (DNA) in pathogens and thereby neutralize their ability to reproduce.

The coronavirus disease 2019 (COVID-19) pandemic has increased the awareness that pathogens (e.g., bacteria, viruses, and fungi) can easily transfer between people by surface contact. Consequently, the usage of UVC ultraviolet disinfection systems in hospitals has rapidly increased, and such UVC ultraviolet disinfection systems are now being deployed in various public locations to mitigate the transfer of pathogens.

Most UVC disinfection systems use high-power low-pressure mercury vapor lamps to produce 254 nanometer (nm) wavelength UVC ultraviolet light. Although different types of pathogens require different dose levels for sufficient disinfection, at least 99.99% of most pathogens can be neutralized with a 254 nm UVC dose of 50 millijoules per square centimeter (mJ/cm²). Consequently, a 50 mJ/cm² dose is often used as the minimum target dose for disinfection.

UVC surface disinfection systems are often configured as a rolling tower containing a dozen or more UVC lamps. One or more towers will generally be rolled into a room and then switched on to illuminate the lamps for a period of time (the room must be unoccupied during disinfection to avoid hazardous UVC dosing of people).

The purpose of such broadcast UVC systems is to disinfect surfaces likely touched by people. However, surfaces such as door handles, light switches, faucets, handrails and the like may represent only 1% of the total surface area in a room. Consequently, 99% of the broadcast UVC energy is simply wasted on surfaces that are not touched. But there is another downside because fabrics in curtains and furniture may be degraded by repeated high UVC exposure.

Shadows represent another challenge for broadcast UVC systems. Many of the surfaces contacted by people are shadowed from a UVC source located in the middle of the room. For instance, the back side of a door handle or handrail will be shadowed and may only receive 2% of the dose received by surfaces directly exposed to the UVC source. If the exposure time for a UVC system is designed to deliver at least 50 mJ/cm² to all exposed surfaces in a room (a common target dose), shadowed surfaces may receive as little as 1 mJ/cm², leaving surface pathogens completely unaffected.

The combination of wasted UVC energy and shadowed contact surfaces results in broadcast UVC systems being both very inefficient and very ineffective. As such, new and improved methods, devices, and systems are needed for UVC disinfection.

### SUMMARY

Disclosed herein are methods, systems, and devices for providing light for surface disinfection. In one embodiment, a device includes a controller, a disinfecting light source electronically coupled with the controller, and a positioning mechanism mechanically coupled with the light source and electronically coupled with the controller. Additionally, the controller is configured to direct the disinfecting light source using the positioning mechanism. In some embodiments, the disinfecting light source may be an ultraviolet-C (UVC) light emitting diode (LED) light source, a UVC mercury vapor lamp light source, and/or the like.

In some embodiments, the device may further include a mounting mechanism mechanically coupled with the positioning mechanism. In further embodiments, the mounting mechanism may be configured to attach the positioning mechanism to an approximately flat surface. In still further embodiments, the approximately flat surface may be at least a portion of a wall, a ceiling, or a moving platform. The moving platform may be a robotic platform. The robotic platform may be an autonomous drone. The robotic platform may be a rolling tower platform.

In some embodiments, the positioning mechanism may be configured for pan and tilt capability of the disinfecting light source.

In some embodiments, the device may further include motion sensor circuitry electronically coupled with the controller and mechanically coupled with the disinfecting light source. In further embodiments, the motion sensor circuitry may include a six-axis inertial measurement unit (IMU). Additionally, the controller may be configured to receive acceleration data and/or rotational data from the motion sensor circuitry, and adjust the positioning mechanism based on the acceleration data and/or the rotational data.

In some embodiments, the device may further include a camera and/or a camera system electrically coupled with the controller. In further embodiments, the camera system may be an infrared (IR) stereo vision camera system configured for determining an approximate distance to an object. In still further embodiments, the device may further include an IR light source electronically coupled with the controller and the IR light source may be configured for providing illumination for the IR stereo vision camera system.

In some embodiments, the device may further include a laser source. The controller may be configured for determining the optical pattern of one or more laser beams produced by the laser source and captured within an image by the camera, and configured for determining an approximate distance to an object based on the diameter of the one or more laser beams.

In some embodiments, the controller may include artificial intelligence (AI) circuitry including machine learning capabilities for processing images and recognizing objects captured by the camera and/or camera system (e.g., machine vision).

In some embodiments, the controller may be configured for receiving images captured by the and/or camera system, determining an object for disinfection based on the images, directing the disinfecting light source toward the object using the positioning mechanism, and activating the disinfecting light source to provide the disinfection of the object. In further embodiments, the controller may be further configured for determining an approximate distance to the object based on the images and determining an activation time and/or a power level for the disinfecting light source based the approximate distance to the object. In further embodiments, the controller may be further configured for detecting motion based on the images and deactivating the disinfecting light source based on detecting the motion.

In some embodiments, the device may further include a communication interface electrically coupled with the controller. The controller may be further configured for transmitting disinfection status to a remote computing system via the communication interface, transmitting the images to the remote computing system, receiving disinfecting light source control information from the remote computing system, and/or receiving positioning mechanism control information from the remote computing system. In further embodiments, the remote computing system may be provided by a cloud computing service.

In some embodiments, the communication interface may be configured for mesh networking between a remote computing system and a plurality of other devices configured for disinfecting surfaces.

In some embodiments, the device may further include an enclosure and the disinfecting light source may be configured to be retractable into the enclosure and hidden when the device is inactive. Additionally, the camera and/or camera system may be retractable to ensure privacy of room occupants when the device is inactive.

In some embodiments, the device may be configured for installation or temporary usage in an area intended for human occupancy.

In another embodiment, a method implemented by a controller on a device for disinfecting surfaces is disclosed. The method includes (1) extending a disinfecting light source using a positioning mechanism that was previously retracted, (2) directing the disinfecting light source towards an object using a pan and tilt capability of the positioning mechanism, (3) activating the disinfecting light source for a predetermined period of time, (4) deactivating the disinfecting light source, and (5) retracting the disinfecting light source. Additionally, the positioning mechanism is mounted to an approximately flat surface.

In another embodiment, a method of manufacturing a device for disinfecting surfaces is disclosed. The method includes (1) electrically coupling a controller with a disinfecting light source, and a positioning mechanism; (2) mechanically coupling the disinfecting light source with the positioning mechanism; and (3) mechanically assembling the controller, the disinfecting light source, and the positioning mechanism within an enclosure. Additionally, the enclosure is configured for (1) mounting to an approximately flat surface, (2) extending the disinfecting light source using the positioning mechanism under command of the controller, and (3) retracting the disinfecting light source using the positioning mechanism under command of the controller.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The present embodiments are illustrated by way of example and are not intended to be limited by the figures of the accompanying drawings. In the drawings:

FIG. 1 depicts a block diagram illustrating a system including a device for providing light for surface disinfection, a remote computing system, and a mobile device in accordance with embodiments of the present disclosure.

FIG. 2 depicts a diagram illustrating an extended view of a preferred embodiment of the device of FIG. 1 during an active disinfection session in accordance with embodiments of the present disclosure.

FIG. 3 depicts a diagram illustrating a retracted view of the preferred embodiment of the device of FIG. 1 during an inactive mode in accordance with embodiments of the present disclosure.

FIG. 4 depicts a block diagram further illustrating the mobile device of FIG. 1 in accordance with embodiments of the present disclosure.

FIG. 5 depicts a block diagram illustrating a server as one embodiment of the remote computing system of FIG. 1 in accordance with embodiments of the present disclosure.

FIG. 6 depicts a flow chart illustrating a method implemented by a controller on a device for disinfecting surfaces in accordance with embodiments of the present disclosure.

FIG. 7 depicts a flow chart illustrating a method of manufacturing a device for disinfecting surfaces in accordance with embodiments of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Disclosed herein are methods, systems, and devices for providing light for surface disinfection. The provided light may be many times more intense (e.g., directed and possibly focused light) than can be provided by disinfection systems using only broadcast light. This higher intensity light means that a sufficient dose can be delivered to targeted areas to achieve dose goals, even with shadows present.

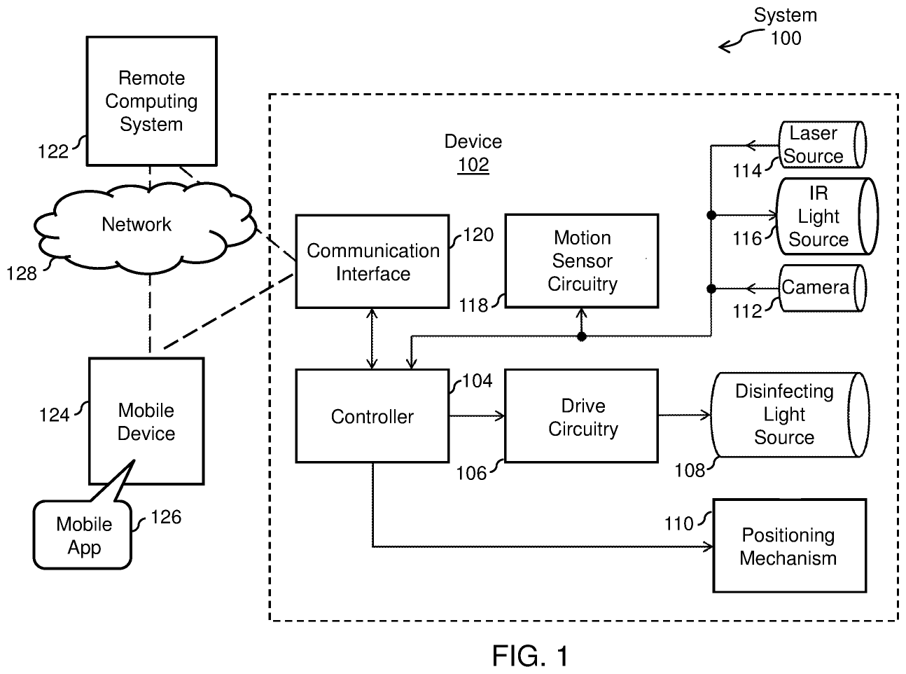

FIG. 1 depicts a block diagram illustrating a system 100 including a device 102 for providing light for surface disinfection in accordance with embodiments of the present disclosure. The device 102 includes a controller 104 electrically coupled with drive circuitry 106, a disinfecting light source 108, a positioning mechanism 110, a camera 112, a laser source 114, an infrared (IR) light source 116, motion sensor circuitry 118, and a communication interface 120. The device 102 also includes an enclosure and a mounting mechanism (not shown in FIG. 1). The device 102 may also be configured for mounting over an alternating current (AC) outlet box (e.g., a single gang box or a double gang box).

The controller 104 may include one or more reduced instruction set computer (RISC) processors. The RISC processors may be based on a 32-bit architecture and/or a 64-bit architecture. The controller 104 may also be single system on chip (SoC) device or a plurality of application specific circuits (ASICs). The ASICs may include one or more image signal processors (ISPs) and artificial intelligence (AI) accelerators. The AI accelerators may include machine learning capabilities and implement neural networks and machine vision for the device 102.

The disinfecting light source 108 may include an ultraviolet-C (UVC) light emitting diode (LED) light source (e.g., a plurality of LEDs) and the drive circuitry 106 may include one or more UVC LED power regulators. In other embodiments, the disinfecting light source 108 may include a UVC mercury vapor lamp light source and the drive circuitry 106 may include one or more mercury vapor lamp ballasts. The disinfecting light source 108 may also include one or more lenses for focusing a beam of light for surface disinfection. The one or more lenses may include one or more reflective lenses and/or one or more refractive lenses. Additionally, the one or more lenses may be adjustable via the controller 104. The drive circuitry 106 may be configured to adjust a power level of the disinfecting light source 108 in addition to activation and deactivation. In certain embodiments, the disinfecting light source 108 may be configured to have a light beam diameter of five inches to ten inches at thirty feet. In further embodiments, the light beam diameter may be approximately eight inches at thirty feet.

The positioning mechanism 110 is mechanically coupled with the disinfecting light source 108 and is configured to direct the disinfecting light source 108 to one or more objects using at least two-axis of motion. The positioning mechanism 110 is positioned under command of the controller 104. The positioning mechanism 110 may be a two-axis turret aiming actuator. The positioning mechanism 110 is further configured to extend and retract the disinfecting light source 108 and/or camera 112 from the enclosure.

The camera 112 is mechanically coupled with the positioning mechanism 110 and the camera 112 is configured to capture images targeted for surface disinfection. The laser source 114 is also mechanically coupled with the positioning mechanism 110 and configured to source at least one laser beam on an object. The controller 104 is configured for determining a diameter of the laser beam within an image captured by the camera 112 and determining an approximate distance to the object based on the diameter of the laser beam pattern within the image.

The IR light source 116 is also mechanically coupled with the positioning mechanism 110 and configured to provide illumination for the camera 112. In certain embodiments, a separate positioning mechanism (not shown in FIG. 1) may be used for aiming the IR light source 116, the camera 112, and the laser source 114. The one or more ISPs may be configured to autocorrect images that may be rotated or distorted based on the two-axis of rotation.

The motion sensor circuitry 118 is mechanically coupled with the positioning mechanism 110 and may be used for rough accuracy aiming of the disinfecting light source 108 and for aligning a targeted object within a field of view of the camera 112. The motion sensor circuitry may be configured to provide acceleration data and/or rotational data to the controller 104. The one or more ISPs may be further configured to use the acceleration data and/or rotational data for autocorrection of images. The motion sensor circuitry 118 may include a six-axis inertial measurement unit (IMU). In other embodiments, the motion sensor circuitry 118 may include a three-axis accelerometer and a three-axis magnetometer.

The system 100 further includes a remote computing system 122 and a mobile device 124. The mobile device 124 is configured to execute a mobile application (app) 126. The device 102 is communicatively coupled with the remote computing system 122 and the mobile device 124 via the network 128. The controller 104 is configured to receive command data and transmit status data via the communication interface 120 to the remote computing system 122. In further embodiments, the controller 104 may transmit image data captured by the camera 112.

In some embodiments, the mobile device 124 may communicate directly with the device 102. In other embodiments, a mobile device 124 may not be used at all by the device 102.

The communication interface 120 of the device 102 may include an Ethernet interface, a wireless personal area network (WPAN) interface (e.g., Bluetooth®), a wireless local area network (WLAN) interface (e.g. 802.11.x), a cellular network interface (e.g. 2G, 3G, 4G, 5G), a Zigbee® network interface, a Zwave® network interface, a LoRaWAN® network interface, and/or the like.

The communication interface 120 may use one or more transfer protocols to communicate with the remote computing system 122 and/or the mobile device 124 over the network 128 The transfer protocols may include a hypertext transfer protocol (HTTP) session, an HTTP secure (HTTPS) session, a secure sockets layer (SSL) protocol session, a transport layer security (TLS) protocol session, a datagram transport layer security (DTLS) protocol session, a file transfer protocol (FTP) session, a user datagram protocol (UDP), a transport control protocol (TCP), a remote direct memory access (RDMA) transfer protocol, or the like.

The network 128 may include one or more WPANs, one or more WLANs, one or more cellular networks, one or more Zigbee® networks, one or more Zwave®, networks, one or more LoRaWAN® networks, and/or the like. The WPANs may include one or more Bluetooth® Low Energy (BLE) networks. The WLANs may include Wi-Fi technologies such as 802.11a, 802.11b/g/n, and/or 802.11ac technologies. The cellular networks may include 2G, 3G, 4G, and/or 5G technologies. The network 128 may also include the Internet.

The controller 104 and the camera 112 are configured to identify specific objects or surfaces that have been programmed for disinfection. Optical image recognition software running on the controller 104 can recognize desired targets for surface disinfection and aim the disinfecting light source 108 using the positioning mechanism 110. For large objects or surfaces, the controller 104 may sweep the disinfecting light source 108 using the positioning mechanism 110 such that sufficient surface disinfection occurs.

The device 102 will generally operate when a room is unoccupied and often when the room is dark. As such, the IR light source 116 is needed such that the camera 112 can view objects with little or no visible light.

Camera 112 may also be used to detect motion near a light beam from the disinfecting light source 108 and the controller 104 can then disable or reposition the disinfecting light source 108 when a human (or animal) may be nearby. This protective response can occur when the target is illuminated by either visible light or IR light. In some embodiments, the device 102 may also include one or more microphones (not shown in FIG. 1). The one or more microphones may also be used to detect human or animal activity. Additionally, the device 102 may include one or more motion sensors (separate from the camera 112 and not shown in FIG. 1) and electrically coupled with the controller 104. The one or more motion sensors may also be used to detect human and/or animal activity. The one of more motion sensors may include one or more passive IR (PIR) detectors.

In some embodiments, the camera 112 may include two cameras (not shown in FIG. 1) and be configured to provide IR stereo vision (including distance calculations using the controller). Such distance information is valuable when mapping the room geometry (useful during programming) in addition to calculating the power at the target so an accurate estimate of a light dose at each target can be calculated.

Programming the system to disinfect various selected objects and surfaces can be done by an operator in the same room as the device 102 or at a remote location. Using mobile device 124, the operator can manually control the aiming of the disinfecting light source 108 while viewing images captured from the cameras 112 on the mobile device 124. Each specific target can be specified along with a desired disinfecting light dose, and the controller 104 can later precisely direct the disinfecting light source 108 based on AI-based image recognition of the target.

AI needed during programming may be provided by the remote computing system 122 and/or directly by the controller 104. AI may dramatically simplify programming the device 102. For example, in a classroom with many identical desks and chairs, the system 100 could be configured on how to disinfect a single desk and chair. Next, AI could be instructed to deliver specified disinfecting light doses to all similar desks and chairs in the classroom.

The device 102 may also include additional sensors (not shown in FIG. 1) for measuring air quality. For example, the additional sensors may be configured to monitor one or more of particulate matter (PM10, PM2.5, PM1, and PM0.1), carbon monoxide, carbon dioxide, ozone, sulfur dioxide, smoke, and heat. The device 102 may also be configured to transmit an alarm status when National Ambient Air Quality Standards (NAAQS) limits are exceeded.

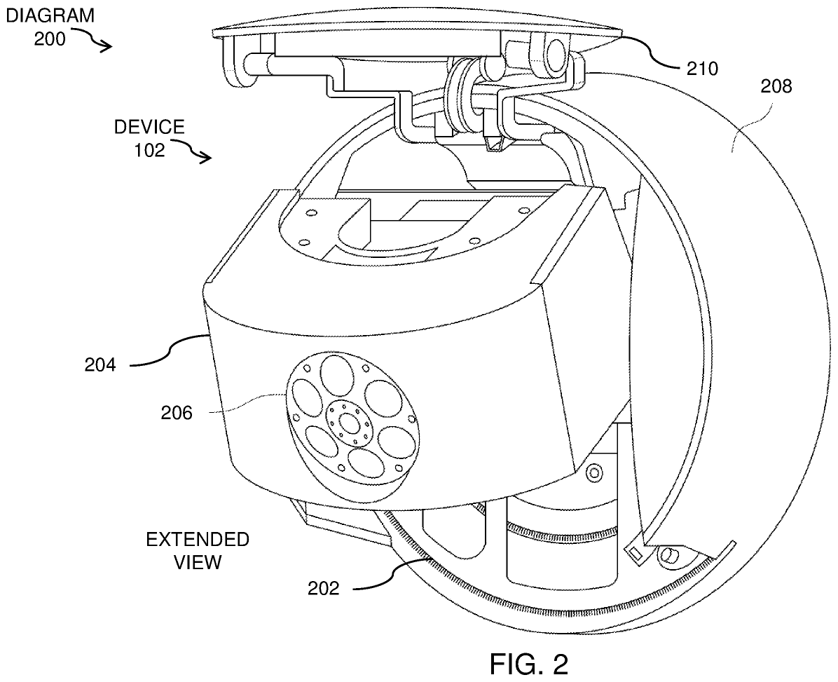

FIG. 2 depicts a diagram 200 illustrating an extended view of a preferred embodiment of the device 102 of FIG. 1 within an enclosure during an active disinfection session in accordance with embodiments of the present disclosure. A mounting mechanism allowing the device 102 to be mounted to an approximately flat service (e.g., a wall, a ceiling, a moving platform, etc.) is located on the rear of the enclosure and not visible in FIG. 2.

As depicted, the positioning mechanism 110 is implemented by combining a rotating turret 202 and a tilting extended face 204 that includes a portal 206. As such the positioning mechanism 110 of FIG. 2 operates in a similar manner to a military tank turret. The disinfecting light source 108, the camera 112, the laser source 114, and the IR light source 116 are exposed via the portal 206. The disinfecting light source 108 utilizes a plurality of UVC LEDs, while the camera 112 and the IR light source 116 provide an internal camera system that allows a UVC beam to be accurately positioned on each selected object or surface while also providing object distance information for correct UVC dose estimation. The object distance information may be determined using the laser source 114 as discussed earlier.

The rotating turret 202 is configured for 360 degrees of mobility and the tilting extended face 204 is configured for 90 degrees of mobility. This mechanical configuration allows a light beam from the disinfecting light source 108 to be aimed at virtually any object within a room. As shown in FIG. 2, the device 102 could be deployed in a minimum of two locations (either ceiling or high on the walls) within a room and provide nearly complete surface disinfection on surfaces typically touched by a human. A rotating skirt 208 and lid 210 is used to conceal and protect the portal 206 and tilting extended face 204 while inactive and in the retracted mode.

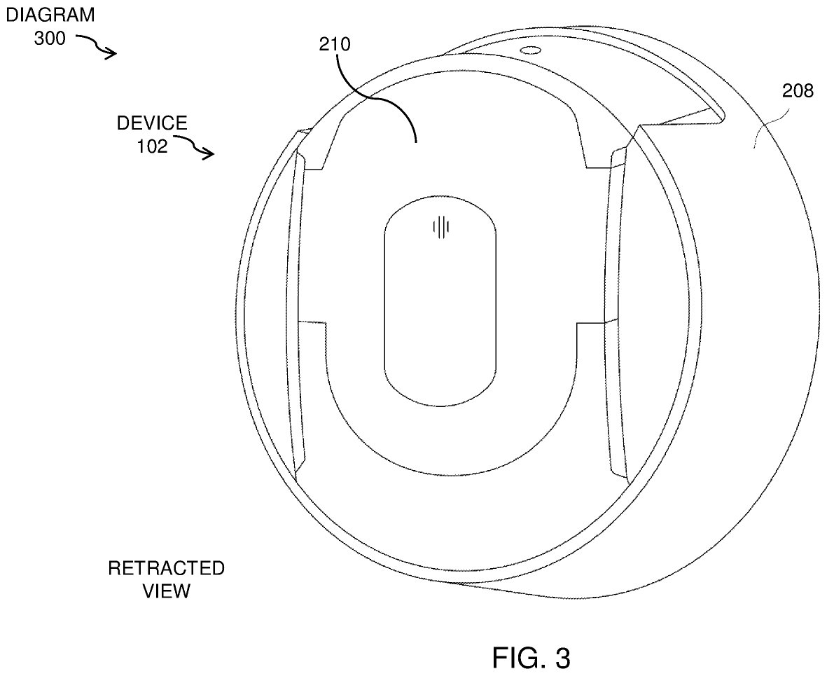

FIG. 3 depicts a diagram 300 illustrating a retracted view of the preferred embodiment of the device 102 of FIG. 2 during an inactive mode in accordance with embodiments of the present disclosure. While retracted, the disinfecting light source 108, the positioning mechanism 110, the camera 112, the laser source 114, and the IR light source 116 are protected from dust and otherwise damage by the lid 210. By hiding the camera 112 while the device 102 is retracted, privacy is assured for room occupants.

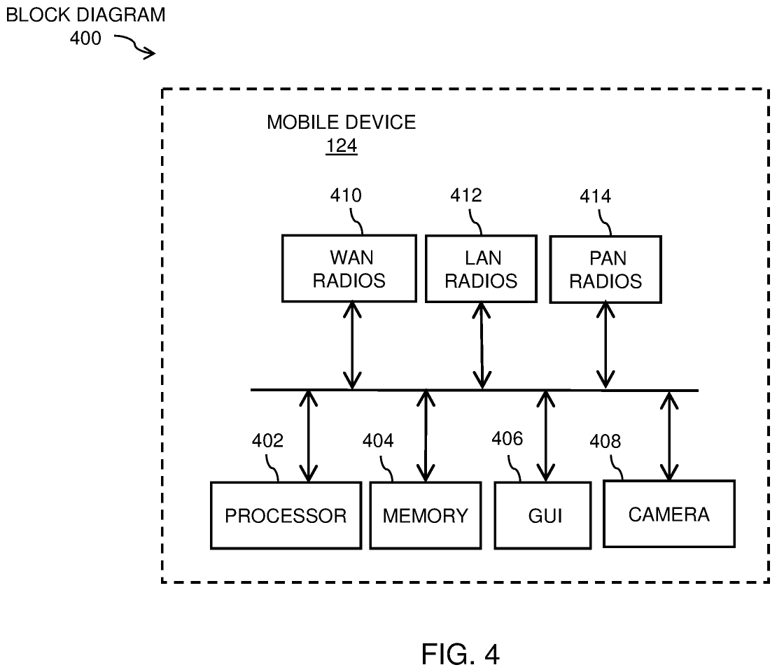

FIG. 4 depicts a block diagram 400 illustrating the mobile device 124 of FIG. 1 in accordance with embodiments of the present disclosure. The mobile device 124 may be a smart phone, a smart tablet, a smart watch, a laptop, or the like. The mobile device 124 may include at least a processor 402, a memory 404, a graphical user interface (GUI) 406, a camera 408, WAN radios 410, LAN radios 412, and PAN radios 414. In some embodiments, the mobile device 2124 may be an iPhone® or an iPad®, using iOS® as an operating system (OS). In other embodiments, the mobile device 124 may be a mobile terminal including Android® OS or the like.

In some embodiments, the processor 402 may be a mobile processor such as the Qualcomm® Snapdragon™ mobile processor. The memory 404 may include a combination of volatile memory (e.g., random access memory) and non-volatile memory (e.g., flash memory). The memory 404 may be partially integrated with the processor 402. The GUI 406 may be a touchpad display. The WAN radios 410 may include 2G, 3G, 4G, and/or 5G technologies. The LAN radios 412 may include Wi-Fi technologies such as 802.11a, 802.11b/g/n, 802.11ac, 802.11.ax and/or the like circuitry. The PAN radios 414 may include Bluetooth® technologies.

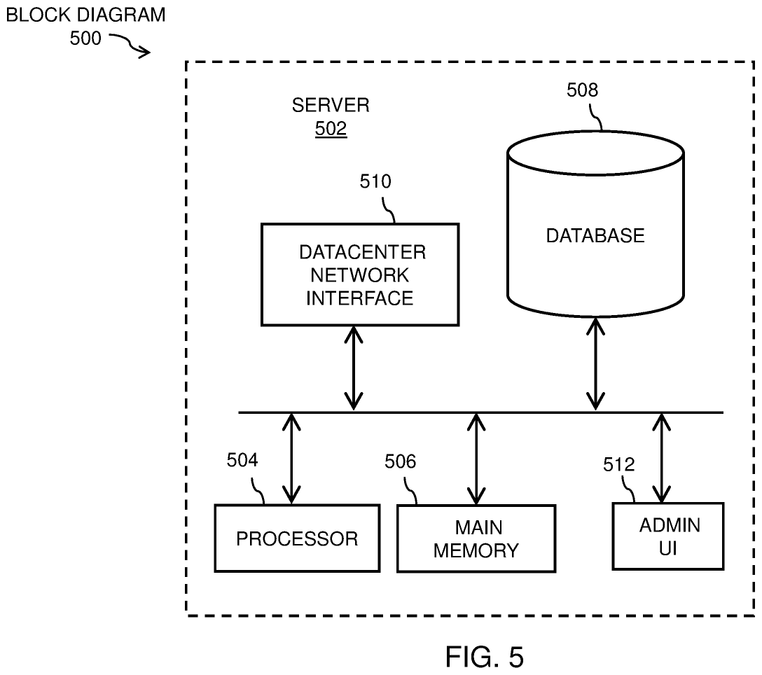

FIG. 5 depicts a block diagram 500 illustrating a server 502 as one embodiment of the remote computing system 122 of FIG. 1 in accordance with embodiments of the present disclosure. The server 502 includes at least one processor 504, a main memory 506, a storage memory (e.g., database) 508, a datacenter network interface 510, and an administration user interface (UI) 512. The server 502 may be configured to host at least a portion of a virtual server (e.g., an Ubuntu® server or the like). In some embodiments, the virtual server may be distributed over a plurality of hardware servers (like the server 502) using hypervisor technology to form the remote computing system 122.

The processor 504 may be a multi-core server class processor suitable for hardware virtualization. The processor may support at least a 64-bit architecture and a single instruction multiple data (SIMD) instruction set. The main memory 506 may include a combination of volatile memory (e.g., random access memory) and non-volatile memory (e.g., flash memory). The database 508 may include one or more hard drives.

The datacenter network interface 510 may provide one or more high-speed communication ports to data center switches, routers, and/or network storage appliances within a cloud computing environment. The datacenter network interface 510 may include high-speed optical Ethernet, InfiniBand (IB), Internet Small Computer System Interface (iSCSI), and/or Fibre Channel interfaces. The administration UI 512 may support local and/or remote configuration of the server 502 by a datacenter administrator.

The server 502 may be implemented within a cloud computing environment such as the Microsoft Azure®, the Amazon Web Services® (AWS), or the like cloud computing data center environments. The server 502 may also be configured to be hosted within a virtual container. For example, the virtual container may be the Docker® virtual container or the like. In some implementations, the virtual container may be distributed over a plurality of hardware servers using hypervisor technology.

The server 502 may also be a computing device implemented within a building where surface disinfection is being completed by a plurality of devices such as device 102. For example, the server 502 may be personal computer (PC), a workstation, and/or the like. The server 502 may also be configured to communicate with one or more additional remote servers.

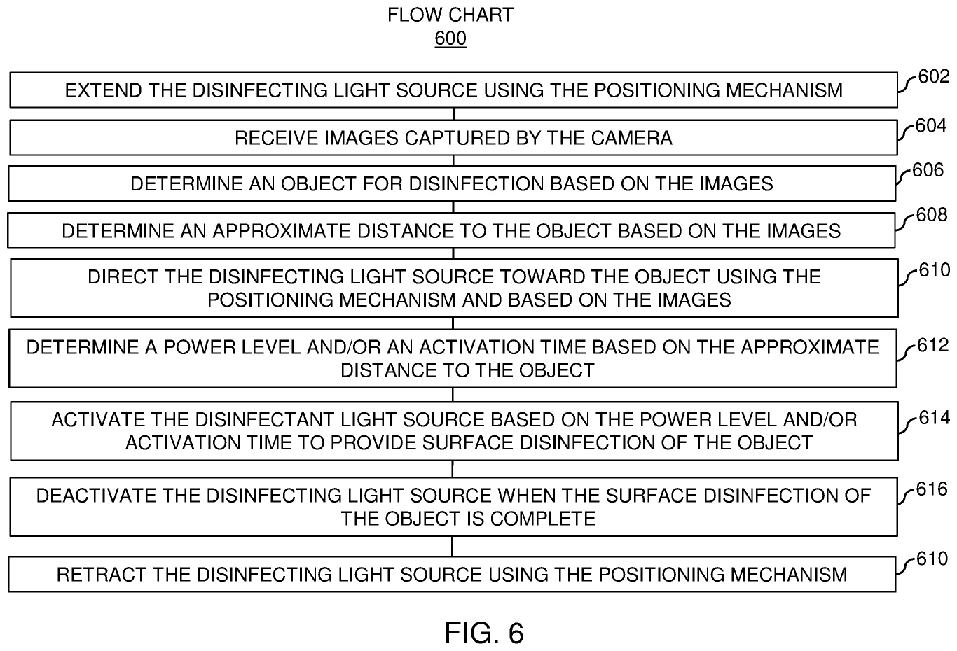

FIG. 6 depicts a flow chart 600 illustrating a method implemented by the controller 104 on the device 102 for disinfecting surfaces of FIG. 1 in accordance with embodiments of the present disclosure.

In step 602, the method includes extending the disinfecting light source 108 using the positioning mechanism 110. The disinfecting light source 108 was previously retracted and inactive prior to step 602.

In step 604, the method further includes receiving images captured by the camera 112. Step 604 may also include activating the IR light source to provide IR illumination for the camera 112.

In step 606, the method further includes determining an object for disinfection based on the images.

In step 608, the method further includes determining an approximate distance to the object based on the images.

In step 610, the method further includes directing the disinfecting light source 108 toward the object using the positioning mechanism 110 and based on information received in the images. Directing the disinfecting light source 108 may include using a pan and tilt capability of the positioning mechanism 110.

In step 612, the method further includes determining a power level and/or an activation time based on the approximate distance to the object

In step 614, the method further includes activating the disinfecting light source 108 based on the power level and/or the activation time to provide surface disinfection of the object. The method (not shown in FIG. 6) may also include receiving additional images captured by the camera 112 detecting motion (e.g., human activity) based on the additional images, and deactivating the disinfecting light source 108 based on detecting the motion. Additionally, the camera 112 may include detecting a human heat signature within the additional images and deactivating the disinfecting light source 108 based on detecting the human heat signature. The method may further include reactivating the disinfecting light source after a predetermined time when the motion is no longer detected and/or the human heat signature is no longer detected.

In step 616, the method further includes deactivating the disinfecting light source 108 when the surface disinfection of the object is complete.

In step 618, the method further includes retracting the disinfecting light source 108 using the positioning mechanism 110. In other embodiments, the method may repeat at step 604, with the device 102 locating and disinfecting the surfaces of additional objects before retracting.

The method may also include receiving a disinfecting schedule from the remote computing system 122 (not shown in FIG. 6). The method of flowchart 600 may then be activated based on the schedule.

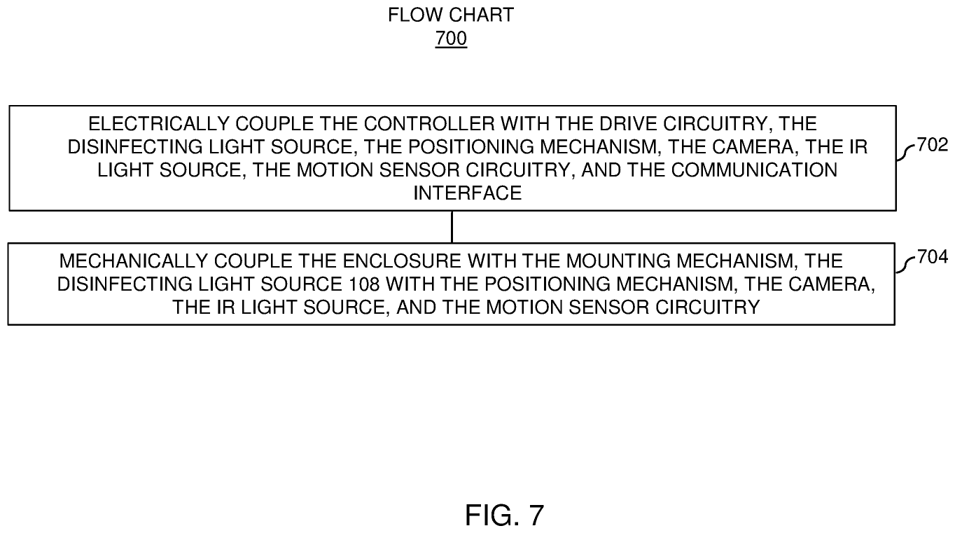

FIG. 7 depicts a flow chart 700 illustrating a method of manufacturing a device for disinfecting surfaces in accordance with embodiments of the present disclosure.

In step 702, the method includes electrically coupling the controller 104 with the drive circuitry 106, the disinfecting light source 108, the positioning mechanism 110, the cameras 112, the laser source 114, the IR light source 116, the motion sensor circuitry 118, and the communication interface 120. One or more printed circuit boards (PCBs) and or wiring harnesses may be used.

In step 704, the method further includes mechanically coupling the enclosure with the mounting mechanism, the disinfecting light source 108 with the positioning mechanism 110, the camera 112, the laser source 114, the IR light source 116, and the motion sensor circuitry 118.

In further embodiments, the device 102 of FIG. 1 may be deployed in pairs within classrooms within a school. A mesh network may be used to backhaul images from the deployed devices to a gateway device. The gateway device including a router and modem may be used to provide connectivity to the remote computing system 122 within a cloud computing center. The deployed devices may be used as a disinfection system and a camera monitoring system. The camera monitoring system may be used by school administrators and/or parents. Other features may include activating classroom door locks during disinfection sessions and after determining no human presence is within the given classroom.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. For example, programming languages may include, but are not limited to: Ruby, JavaScript, Java, Python, Ruby, PHP, C, C++, C#, Objective-C, Go, Scala, Swift, Kotlin, OCaml, or the like.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A device for disinfecting surfaces, the device comprising:
a controller;
a disinfecting light source electronically coupled with the controller; and
a positioning mechanism mechanically coupled with the disinfecting light source and electronically coupled with the controller, wherein the controller is configured to direct the disinfecting light source toward an object using the positioning mechanism.

2. The device of claim 1 further comprising a mounting mechanism mechanically coupled with the positioning mechanism.

3. The device of claim 2, wherein the mounting mechanism is configured to attach the positioning mechanism to an approximately flat surface.

4. The device of claim 3, wherein the approximately flat surface is at least a portion of a wall, a ceiling, or a moving platform.

5. The device of claim 1, wherein the positioning mechanism is configured for pan and tilt capability.

6. The device of claim 1 further comprising motion sensor circuitry electronically coupled with the controller and mechanically coupled with the disinfecting light source.

7. The device of claim 6 wherein the motion sensor circuitry includes a six-axis inertial measurement unit (IMU).

8. The device of claim 1 further comprising a camera electrically coupled with the controller.

9. The device of claim 8 further comprising a laser source, wherein the controller is configured for:
determining a diameter of a laser beam pattern produced by the laser source and captured within an image by the camera; and
determining an approximate distance to an object based on the diameter of the laser beam pattern within the image.

10. The device of claim 9 further comprising an infrared (IR) light source electronically coupled with the controller and configured for providing illumination for the camera.

11. The device of claim 8, wherein the controller comprises artificial intelligence (AI) processing including machine learning for processing images and recognizing objects captured by the camera.

12. The device of claim 8, wherein the controller is configured for:
receiving images captured by the camera;
determining an object for surface disinfection based on the images;
directing the disinfecting light source toward the object using the positioning mechanism; and
activating the disinfecting light source to provide disinfection of the object.

13. The device of claim 12, wherein the controller is further configured for:
determining an approximate distance to the object based on the images; and
determining at least one of an activation time and a power level for the disinfecting light source based on the approximate distance to the object.

14. The device of claim 12, wherein the controller is further configured for:
detecting motion based on the images; and
deactivating the disinfecting light source based on detecting the motion.

15. The device of claim 12 further a communication interface electrically coupled with the controller and the controller is further configured for:
transmitting disinfection status to a remote computing system via the communication interface;
transmitting the images to the remote computing system;
receiving disinfecting light source control information from the remote computing system; and
receiving positioning mechanism control information from the remote computing system.

16. The device of claim 15, wherein the communication interface is configured for mesh networking between the remote computing system and a plurality of other devices configured for disinfecting surfaces.

17. The device of claim 1, wherein the disinfecting light source is at least one of an ultraviolet-C (UVC) light emitting diode (LED) light source and a UVC mercury vapor lamp light source.

18. The device of claim 1 further comprising an enclosure; wherein the disinfecting light source is configured to be retractable into the enclosure and hidden when the device is inactive.

19. The device of claim 1, wherein the device is configured for installation or temporary usage in an area intended for human occupancy.

20. A method implemented by a controller on a device for disinfecting surfaces, the method comprising:
extending a disinfecting light source using a positioning mechanism, wherein the disinfecting light source was previously retracted;
directing the disinfecting light source towards an object using a pan and tilt capability of the positioning mechanism.
activating the disinfecting light source for a predetermined period of time;
deactivating the disinfecting light source; and
retracting the disinfecting light source; wherein the positioning mechanism is mounted to an approximately flat surface.

21. A method of manufacturing a device for disinfecting surfaces, the method comprising:
electrically coupling a controller with a disinfecting light source, and a positioning mechanism;
mechanically coupling the disinfecting light source with the positioning mechanism; and
mechanically assembling the controller, the disinfecting light source, and the positioning mechanism, within an enclosure, wherein the enclosure is configured for:
mounting to an approximately flat surface;
extending the disinfecting light source using the positioning mechanism under command of the controller; and
retracting the disinfecting light source using the positioning mechanism under command of the controller.
